(19) United States
(12) Patent Application Publication
(10) Pub. No.: US 2009/0088788 A1
(43) Pub. Date: Apr. 2, 2009

(54) METHODS AND APPARATUS HAVING MULTIPLE SEPARATELY ACTUATABLE EXPANDABLE MEMBERS

(76) Inventor: Steven MOUW, Santa Clara, CA (US)

Correspondence Address: HAYNES AND BOONE, LLP;IP Section, 2323 Victory Avenue, Suite 700, Dallas, TX, 75219, US

(21) Appl. No.: 11/863,816

(22) Filed: Sep. 28, 2007

(51) Int. Cl. A61M 29/00; A61M 31/00

(52) U.S. Cl. 606/192; 604/508

(57) ABSTRACT

An apparatus includes a first expandable member and a second expandable member. The first expandable member has a collapsed configuration and an expanded configuration, and is configured to displace a portion of a bone when moved from its collapsed configuration to its expanded configuration. The second expandable member has a collapsed configuration and an expanded configuration, and is configured to limit movement of the first expandable member within a body of a patient when in its expanded configuration.

### BACKGROUND

The invention relates generally to medical devices and procedures, including for example, to apparatus having multiple separately actuatable expandable members for displacing tissue within a body of a patient.

Known medical devices employ expandable members to repair fractures and/or other defects of the spinal column. For example, some known catheters include an inflatable balloon configured to be disposed within a bone structure to displace a portion of the bone structure when inflated. A need exists for a medical device and methods for more efficiently maintaining a position of an expandable member when the medical device is in use. A need also exists for a medical device having separately actuatable expandable members.

### SUMMARY

Apparatus and methods for using expandable members in surgical procedures are described herein. In some embodiments, an apparatus includes a first expandable member and a second expandable member. The first expandable member has a collapsed configuration and an expanded configuration, and is configured to displace a portion of a bone when moved from its collapsed configuration to its expanded configuration. The second expandable member has a collapsed configuration and an expanded configuration, and is configured to limit movement of the first expandable member within a body of a patient when in its expanded configuration.

### BRIEF DESCRIPTION OF THE DRAWINGS

FIGS. 1-3 are schematic illustrations of a medical device according to an embodiment of the invention disposed within a bone structure in a first configuration, a second configuration and a third configuration, respectively.

FIG. 4-7 are cross-sectional side views of a medical device from a transverse plane, according to an embodiment of the invention disposed within a vertebra in a first configuration, a second configuration, a third configuration and a fourth configuration, respectively.

FIG. 8 is a side view of a medical device according to an embodiment of the invention.

FIG. 9 is a cross-sectional view of the medical device illustrated in FIG. 8 taken along line X-X in FIG. 8.

FIG. 10 is a cross-sectional view of the medical device illustrated in FIG. 8 taken along line X′-X′ in FIG. 8.

FIGS. 11-13 are partial cross-sectional side views of a medical device according to an embodiment of the invention in a first configuration, a second configuration and a third configuration, respectively.

FIG. 14 is a flow chart illustrating a method for displacing a portion of a tissue according to an embodiment of the invention.

FIG. 15 is a flow chart illustrating a method for displacing a portion of a bone structure according to an embodiment of the invention.

FIG. 16 is a schematic illustration of a medical device according to an embodiment of the invention disposed within a bone structure.

FIG. 17 is a schematic illustration of a medical device according to an embodiment of the invention having three expandable members disposed within a bone structure.

FIG. 18 is a schematic illustration of a medical device according to an embodiment of the invention disposed within a bone structure.

FIG. 19 is a schematic illustration of a medical device according to an embodiment of the invention having a first expandable member disposed within a second expandable member.

FIGS. 20-22 are schematic illustrations a medical device according to an embodiment of the invention having a first shaft movable relative to a second shaft, in a first configuration, a second configuration and a third configuration, respectively.

FIG. 23 is a partial cross-sectional side view of a medical device having a first shaft movable relative to a second shaft according to an embodiment of the invention.

FIGS. 24-28 are cross-sectional side views of a medical device from a transverse plane, according to an embodiment of the invention disposed within a disc in a first configuration, a second configuration, a third configuration, a fourth configuration, and a fifth configuration, respectively.

FIG. 29 is a flow chart illustrating a method for implanting a disc replacement material and/or an implant according to an embodiment of the invention.

FIGS. 30 and 31 are cross-sectional side views of a medical device from a sagittal plane, according to an embodiment of the invention disposed within a disc in a first configuration and a second configuration, respectively.

### DETAILED DESCRIPTION

Apparatus and methods for using expandable members in surgical procedures are described herein. In some embodiments, an apparatus includes a first expandable member and a second expandable member. The first expandable member has a collapsed configuration and an expanded configuration, and is configured to displace a portion of a bone when moved from its collapsed configuration to its expanded configuration. The second expandable member has a collapsed configuration and an expanded configuration, and is configured to limit movement of the first expandable member within a body of a patient when in its expanded configuration.

In some embodiments, an apparatus includes a catheter, a first expandable member and a second expandable member. The first expandable member is coupled to the catheter. The first expandable member has a collapsed configuration and an expanded configuration, and is configured to displace a portion of a bone when moved from its collapsed configuration to its expanded configuration. The second expandable member is coupled to the catheter. The second expandable member has a collapsed configuration and an expanded configuration, and is configured to limit movement of the first expandable member within a body of a patient when in its expanded configuration. The second expandable member is configured to be expanded independently from the first expandable member.

In some embodiments, an apparatus includes a catheter, a first expandable member and a second expandable member. The first expandable member is coupled to the catheter in a first longitudinal position. The first expandable member has a collapsed configuration and an expanded configuration, and is configured to displace a portion of a bone when moved from its collapsed configuration to its expanded configuration. The second expandable member is coupled to the catheter in a second longitudinal position. The second expandable member has a collapsed configuration and an expanded configuration, and is configured to limit movement of the first expandable member within a body of a patient when in its expanded configuration. The second expandable member is configured to be expanded independently from the first expandable member. The catheter is configured to selectively adjust a distance between the first longitudinal position and the second longitudinal position.

In some embodiments, an apparatus includes a catheter defining an inflation lumen, a first expandable member, a second expandable member and a valve. The first expandable member defines an interior region. The second expandable member defines an interior region and has a collapsed configuration and an expanded configuration. The interior region of the second expandable member is fluidically isolated from the interior region of the first expandable member. The second expandable member is configured to limit movement of the first expandable member within a body of a patient when in its expanded configuration. The valve is configured to selectively place the inflation lumen in fluid communication with the interior region of the first expandable member and the interior region of the second expandable member while maintaining fluid isolation between the interior region of the first expandable member and the second expandable member.

In some embodiments, a method includes inserting into a body a catheter having a first expandable member and a second expandable member. The first expandable member is moved from a collapsed configuration to an expanded configuration such that the first expandable member limits movement of the second expandable member relative to the body. The second expandable member is moved from a collapsed configuration to an expanded configuration such that the second expandable member displaces a portion of a tissue, such as, for example, an endplate of a vertebra.

In some embodiments, a method includes inserting into a body a catheter having a first expandable member and a second expandable member. The first expandable member is moved from a collapsed configuration to an expanded configuration such that the first expandable member contacts a first portion of a bone structure. The second expandable member is moved relative to the first expandable member after the moving the first expandable member. For example, in some embodiments, the second expandable member can be moved relative to the first expandable member along a longitudinal axis of the catheter. The second expandable member is moved from a collapsed configuration to an expanded configuration such that the second expandable member displaces a second portion of the bone structure.

In some embodiments, a kit includes a catheter and a trocar. The catheter has a first expandable member and a second expandable member. The first expandable member is configured to displace a first portion of a bone structure when moved from a collapsed configuration to an expanded configuration. The second expandable member is configured to engage a second portion of the bone structure when in an anchor configuration such that movement of the first expandable member within a body of a patient is limited. The trocar is configured to define a passageway within the body through which the catheter can be inserted to access the bone structure. In some embodiments, the kit can further include a cannula configured to be disposed within the passageway and configured to receive at least a portion of the catheter.

As used in this specification and the appended claims, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the words “proximal” and “distal” refer to direction closer to and away from, respectively, an operator (e.g., surgeon, physician, nurse, technician, etc.) who would insert the medical device into the patient, with the tip-end (i.e., distal end) of the device inserted inside a patient's body first. Thus, for example, the end of a medical device first inserted inside the patient's body would be the distal end, while the opposite end of the medical device (e.g., the end of the medical device being operated by the operator) would be the proximal end of the medical device.

The term “expandable member” as used herein includes a component of a medical device that is configured to be changed or moved from a collapsed configuration to an expanded configuration in which a size of the expandable member is larger than the size when the expandable member is in the collapsed configuration. In some variations, for example, an expandable member can be configured to be moved from a collapsed configuration to an expanded configuration by introducing a medium such as liquid and/or gas into the interior of the expandable member. The expandable member can be, for example, a balloon configured to be moved from a collapsed configuration to an expanded configuration. In some applications, the balloon is constructed, at least in part, from a low-compliant material. In other variations, for example, an expandable member can be a substantially solid member configured to expand under certain conditions (e.g., a member constructed from a shape-memory material, a compressed elastomer or the like). In yet other variations, an expandable member can include a deformable member configured to deform under certain conditions (e.g., when an axial load is applied to the expandable member) to move the expandable member from a collapsed configuration to an expanded configuration. In yet other variations, an expandable member can include at least one member and an actuator configured to move, displace and/or deform the member to move the expandable member from a collapsed configuration to an expanded configuration.

FIGS. 1-3 are schematic illustrations of a medical device 100 according to an embodiment of the invention disposed within a bone structure B in a first configuration, a second configuration and a third configuration, respectively. The medical device 100 includes a catheter 102, a first expandable member 120 and a second expandable member 130. The first expandable member 120 is coupled to the catheter 102 at a first position along the longitudinal axis A_{L} of the catheter 102. The second expandable member 130 is coupled to the catheter 102 at a second position along the longitudinal axis A_{L} of the catheter 102 such that the first expandable member 120 and the second expandable member 130 are spaced apart by a distance D. In this manner, the first expandable member 120 can be positioned adjacent a first portion B1 of the bone structure B, and the second expandable member 130 can be positioned adjacent a second portion B2 of the bone structure B. The bone structure B can be, for example, a vertebral body.

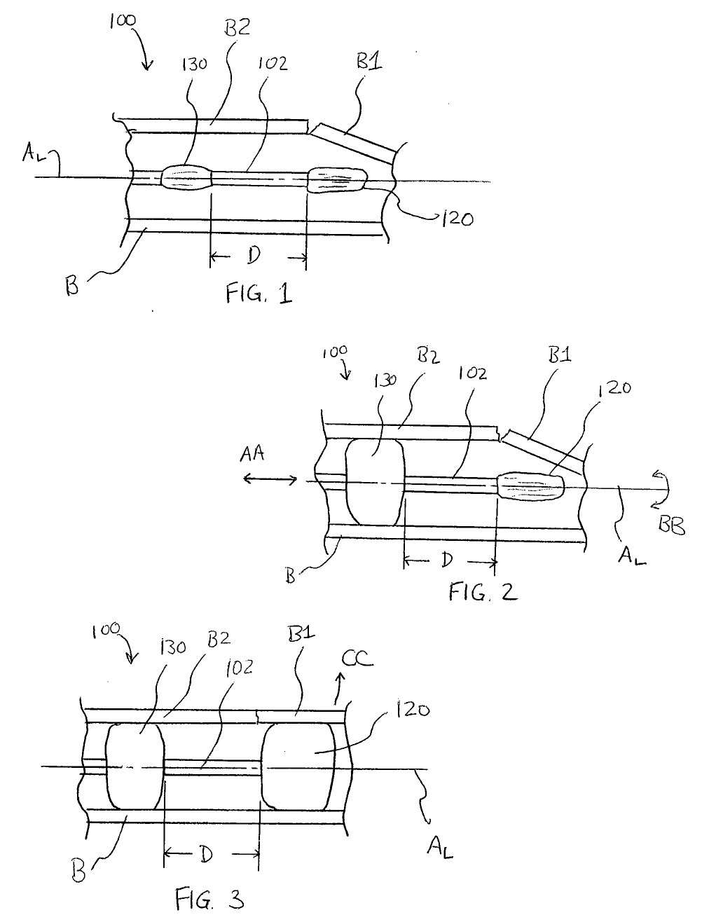

The second expandable member 130, which can be, for example, an inflatable balloon, has a collapsed configuration (see FIG. 1) and an expanded configuration (see FIGS. 2 and 3). When the second expandable member 130 moves from its collapsed configuration to its expanded configuration, a portion of the second expandable member 130 contacts and/or engages the second portion B2 of the bone structure B. In this manner, the second expandable member 130 can anchor the medical device 100 within the bone structure B. Said another way, when the second expandable member 130 is in its expanded configuration, the second expandable member 130 can limit movement of the first expandable member 120 within the bone structure B. More particularly, when the second expandable member 130 is in its expanded configuration and in contact with the second portion B2 of the bone structure B, the second expandable member 130 can limit longitudinal movement (as shown by the arrow AA in FIG. 2) and/or rotational movement (as shown by the arrow BB in FIG. 2) of the first expandable member 120 relative to the bone structure B. For example, in some embodiments, the second expandable member 130 can limit rotational movement of the first expandable member 120 relative to the bone structure B about the longitudinal axis A_{L} as shown by the arrow BB in FIG. 2. Although the second expandable member 130 is shown as anchoring the medical device 100 to limit translation along the longitudinal axis A_{L} and/or rotation about the longitudinal axis A_{L}, in other embodiments, the second expandable member 130 can limit translation and/or rotation along and/or about any axis.

The first expandable member 120, which can be, for example, an inflatable balloon, has a collapsed configuration (see FIGS. 1 and 2) and an expanded configuration (see FIG. 3). When the first expandable member 120 moves from its collapsed configuration to its expanded configuration, a portion of the first expandable member 120 contacts and/or engages the first portion B1 of the bone structure B thereby displacing the first portion B1 as indicated by the arrow CC in FIG. 3.

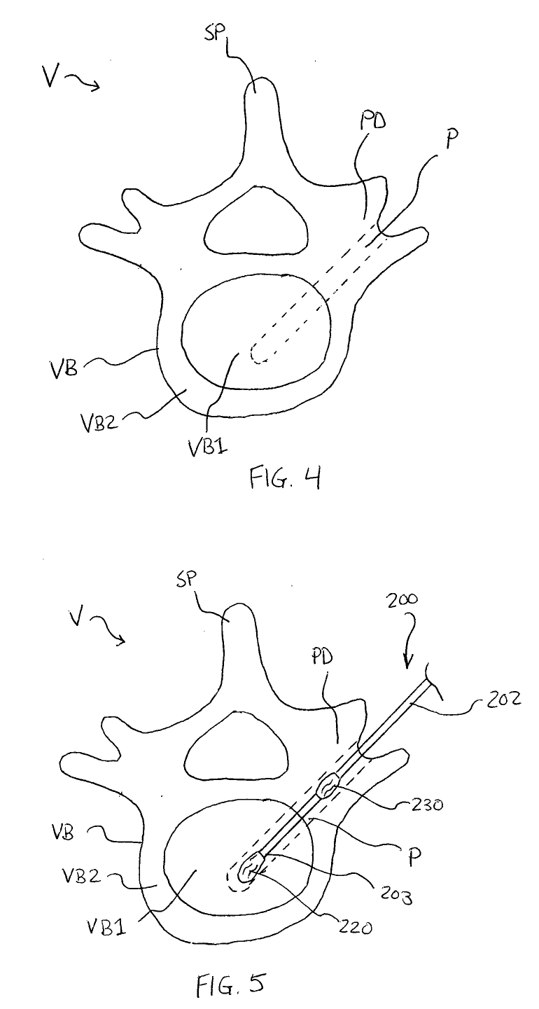

Although the first expandable member 120 is shown as displacing the first portion B1 of the bone structure B by moving the first portion B1, in some embodiments, an expandable member can displace a portion of a bone structure by compacting and/or compressing a portion of the bone structure (e.g., a cancellous bone portion). For example, FIGS. 4-7 are cross-sectional side views of a medical device 200 from a transverse plane, according to an embodiment of the invention disposed within a vertebra V. The medical device 200 includes a catheter 202, a first expandable member 220 disposed at a distal end 203 of the catheter 202, and a second expandable member 230 disposed proximally from the first expandable member 220. The vertebra V includes a spinous process SP, a pedicle PD and a vertebral body VB. The vertebral body VB includes an inner portion VB1 formed from cancellous bone disposed within an outer portion VB2 formed from a cortical bone.

As shown in FIG. 4, an access passageway P can be defined to allow the medical device 200 to be inserted into the vertebral body VB. The passageway P can have any suitable shape (e.g., linear, curved in a single plane, curved in multiple planes or the like) and can be formed by any suitable tool, such as, for example, a trocar (not shown in FIGS. 4-7). A portion of the passageway P can be disposed within the pedicle PD to provide access to the inner portion VB1 of the vertebral body VB.

As shown in FIG. 5, the medical device 200 can be inserted into the body such that the second expandable member 230 is disposed within the passageway P adjacent the pedicle PD and the first expandable member 220 is disposed within the cancellous inner portion VB1 of the vertebral body VB. As shown in FIG. 6, the second expandable member 230 can be moved from its collapsed configuration to its expanded configuration such that the second expandable member 230 contacts a portion of the pedicle PD and/or a portion of the outer portion VB2 of the vertebral body VB. Said another way, when the second expandable member 230 is in its expanded configuration, a portion of the second expandable member 230 contacts the inner surface of the passageway P. In this manner, when the second expandable member 230 is in the expanded configuration, the second expandable member 230 can maintain a location and/or orientation of the medical device 200 within the vertebral body VB. Said another way, when the second expandable member 230 is in the expanded configuration, the second expandable member 230 can anchor the medical device 200 within the vertebral body VB.

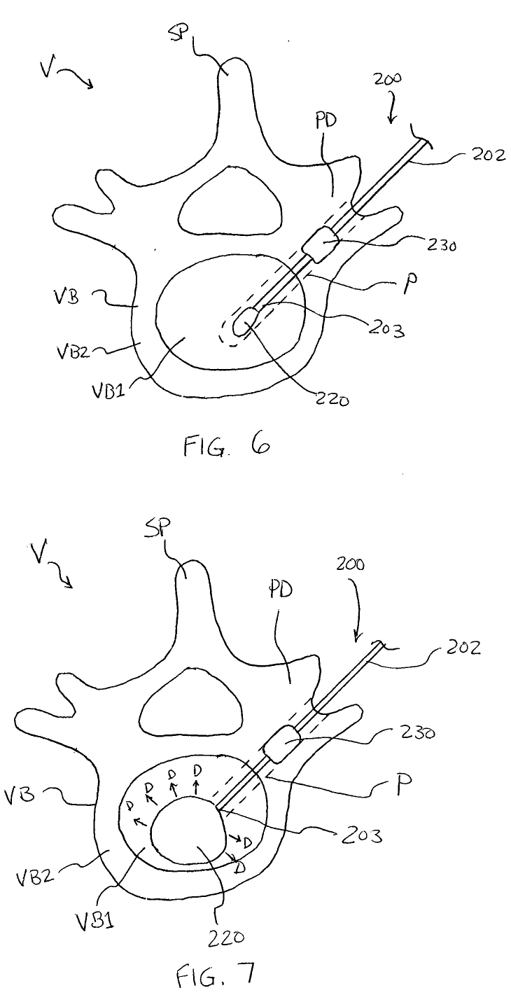

The first expandable member 220 can then be moved from its collapsed configuration to its expanded configuration, as shown in FIG. 7. In this manner, the first expandable member 220 can move a portion of the inner portion VB1 (e.g., the cancellous bone) of the vertebral body VB by compacting and/or compressing the inner portion VB1 as indicated by the arrows DD in FIG. 7. The first expandable member 220 can also move a portion of the inner portion VB1 of the vertebral body VB in other directions (e.g., into and/or out of the page with respect to FIG. 7).

Although the first expandable members 120 and 220 are shown as displacing a portion of a bone structure by moving and/or compacting that portion, respectively, in some embodiments, an expandable member can displace multiple portions of a bone structure by compacting and/or moving those portions. For example, in some embodiments, an expandable member can compact a cancellous portion of a vertebral body and move an endplate of a vertebral body.

As described above, in some embodiments, a second expandable member (e.g., an “anchoring” balloon) can be moved from its collapsed configuration to its expanded configuration before a first expandable member (e.g., a bone displacement balloon) is moved from its collapsed configuration to its expanded configuration. In this manner, the location and/or orientation of the first expandable member can be maintained before the first expandable member is expanded to displace a portion of a bone structure. Similarly, stated, in some embodiments, the first expandable member and the second expandable member can be actuated (i.e., moved between their respective expanded and collapsed configurations) independently from each other. To accommodate such independent actuation, in some embodiments, a medical device can include a catheter having multiple inflation lumens.

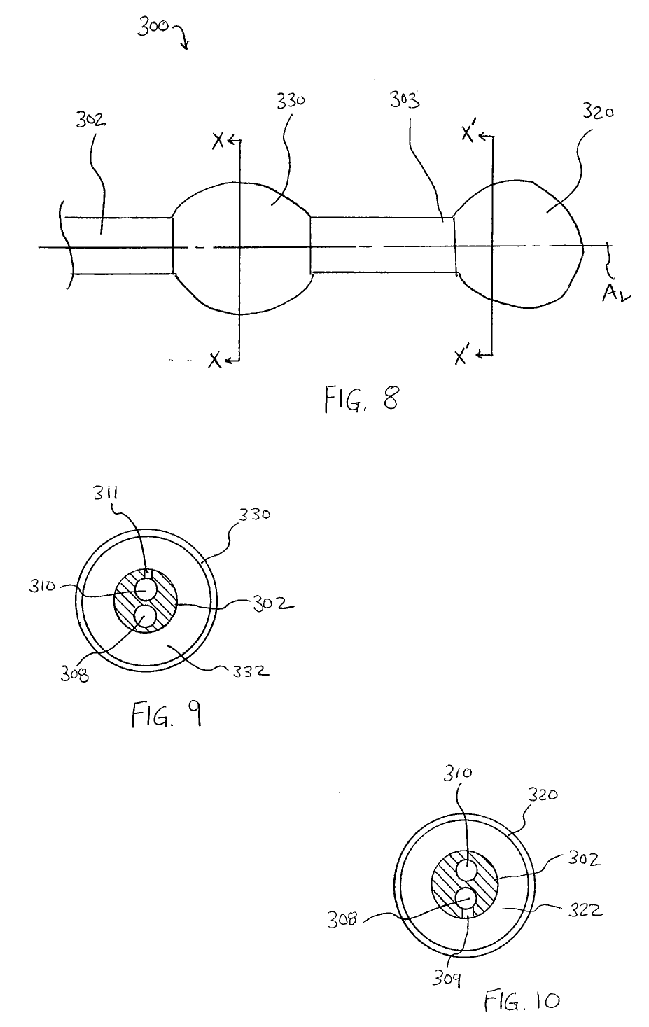

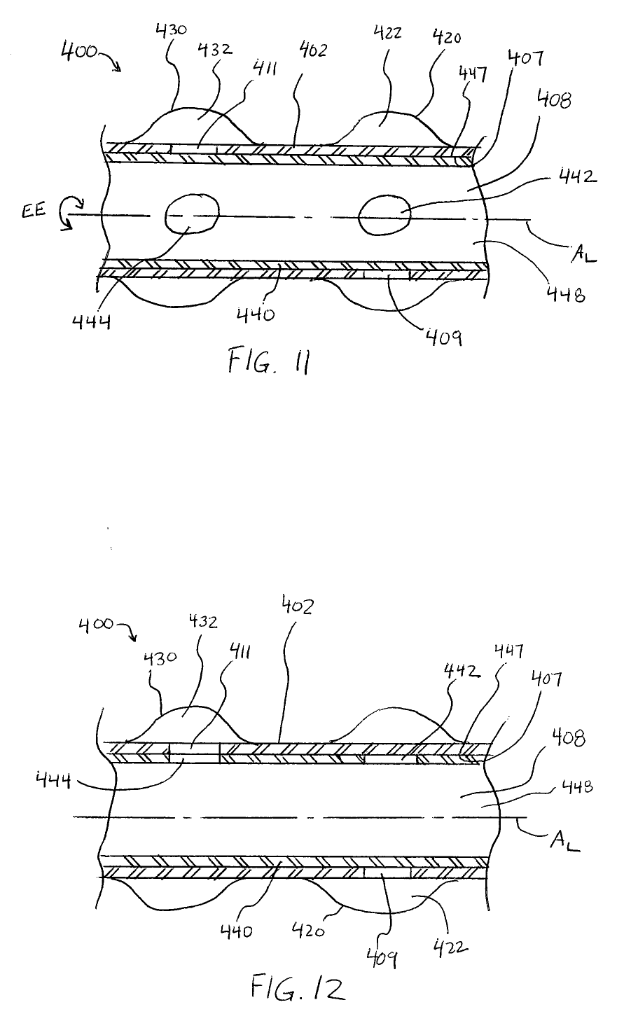

For example, FIGS. 8-10 show a medical device 300 according to an embodiment of the invention. The medical device 300 includes a catheter 302, a first expandable member 320 disposed at the distal end 303 of the catheter 302, and a second expandable member 330 spaced proximally along the longitudinal axis A_{L} from the first expandable member 320. As described above, in some embodiments, the first expandable member 320 can be configured to engage a portion of a bone structure to move, displace, compact and/or compress the portion of the bone structure. In some embodiments, the second expandable member 330 can be configured to contact and/or engage a portion of a body to maintain a location and/or orientation of the catheter 302 within the body. The first expandable member 320 defines an interior region 322 (see e.g., FIG. 10) within which an inflation medium, such as for example, saline solution, can be conveyed to move the first expandable member 320 from its collapsed configuration to its expanded configuration. The second expandable member 330 defines an interior region 332 (see e.g., FIG. 9) within which an inflation medium can be conveyed to move the second expandable member 330 from its collapsed configuration to its expanded configuration. The interior region 322 of the first expandable member 320 is fluidically isolated from the interior region 332 of the second expandable member 330.

The catheter 302 defines a first inflation lumen 308 and a second inflation lumen 310 fluidically isolated from the first inflation lumen 308. As shown in FIG. 10, the first inflation lumen 308 is in fluid communication with the interior region 322 of the first expandable member 320 via an opening 309. As shown in FIG. 9, the second inflation lumen 310 is in fluid communication with the interior region 332 of the second expandable member 330 via an opening 311. In this manner, the first expandable member 320 and the second expandable member 330 can be moved between their respective collapsed and expanded configurations independently from each other. Moreover, this arrangement allows the first expandable member 320 and the second expandable member 330 to be filled with different inflation mediums, maintained at different pressures, and the like.

In some embodiments, the inflation medium can be conveyed to the interior region 322 of the first expandable member 320 and the interior region 332 of the second expandable member 330 from a single source. For example, in some embodiments, a reservoir (not shown in FIGS. 8-10) can be fluidically coupled to the first inflation lumen 308 and the second inflation lumen 310 via one or more valves (not shown in FIGS. 8-10) configured to selectively place the reservoir in fluid communication with the first expandable member 320 and/or the second expandable member 330.

Although the catheter 302 is shown and described having two fluidically isolated inflations lumens (inflation lumen 308 and inflation lumen 310), in some embodiments, the inflation medium can be conveyed to a first expandable member and a second expandable member via a single passageway. For example, FIGS. 11-13 are partial cross-sectional views of a medical device 400 according to an embodiment of the invention. The medical device 400 includes a catheter 402, a first expandable member 420 coupled to the catheter 402, a second expandable member 430 coupled to the catheter 402 spaced apart from the first expandable member 420 along the longitudinal axis A_{L}, and a valve 440. The first expandable member 420 defines an interior region 422 within which an inflation medium can be conveyed to move the first expandable member 420 from its collapsed configuration to its expanded configuration. The second expandable member 430 defines an interior region 432 within which an inflation medium can be conveyed to move the second expandable member 430 from its collapsed configuration to its expanded configuration.

The catheter 402 has an inner surface 407 that defines a lumen 408, a first opening 409, and a second opening 411. The first opening 409 is disposed along the boundary of the interior region 422 of the first expandable member 420. Similarly, the second opening 411 is disposed along the boundary of the interior region 432 of the second expandable member 430. The second opening 411 is spaced apart from the first opening 409 along the circumference of the catheter 402. Said another way, the second opening 411 is offset from the first opening 409 by approximately 180 degrees along the circumference of the catheter 402. Although the second opening 411 is shown as being spaced approximately 180 degrees apart from the first opening 409 along the circumference of the catheter 402, in other embodiments, the second opening 411 can be spaced circumferentially from the first opening 409 by any suitable angle. In yet other embodiments the second opening 411 can be angularly aligned with the first opening 409 along the circumference of the catheter 402 (e.g., the offset between the first opening 409 and the second opening 411 can be zero degrees).

The valve 440 includes an inflation lumen 448, a first port 442 and a second port 444. The valve 440 is disposed within the lumen 408 of the catheter 402 such that when the valve is in its first position, as shown in FIG. 11, an outer surface 447 of the valve 440 and the inner surface 407 of the catheter 402 form a substantially fluid-tight seal about the first opening 409 and the second opening 411. Similarly stated, when the valve 440 is in its first position, a portion of the outer surface 447 of the valve 440 is disposed about the first opening 409 of the catheter 402 and the second opening 411 of the catheter 402 such that the first opening 409 and the second opening 411 are fluidically isolated from the inflation lumen 448 of the valve 440. In this manner, the interior region 422 of the first expandable member 420 and the interior region 432 of the second expandable member 430 can be maintained in fluid isolation from each other. Moreover, this arrangement allows an inflation medium to be conveyed to the interior region 422 of the first expandable member 420 and the interior region 432 of the second expandable member 430 via a common inflation lumen 448.

The first port 442 of the valve 440 is disposed along the longitudinal axis A_{L} in longitudinal alignment with the first opening 409 of the catheter 402. The second port 444 of the valve 440 is spaced apart from the first port 442 along the longitudinal axis A_{L} and is in longitudinal alignment with the second opening 411 of the catheter 402. As shown in FIG. 11, the second port 444 of the valve 440 is angularly aligned with the first port 442 along the circumference of the valve 440 (e.g., the offset between the first port 442 and the second port 444 is zero degrees). As described in more detail herein, the circumferential arrangement of the first port 442, the second port 444, the first opening 409 and the second opening 411 is such that the first expandable member 420 can be moved between its collapsed configuration and its expanded configuration independently from when the second expandable member 430 is moved between its collapsed configuration and its expanded configuration. Although the first port 442 and the second port 444 are shown as being angularly aligned along the circumference of the valve 440, in other embodiments, the second port 444 can be offset circumferentially from the first port 442 by any suitable angle.

In use, the valve 440 can be rotated within the catheter 402 as indicated by the arrow EE in FIG. 11 between a first position (FIG. 11), a second position (FIG. 12) and a third position (FIG. 13). As described above, when the valve 440 is in the first position, the outer surface 447 of the valve 440 is disposed about the first opening 409 of the catheter 402 and the second opening 411 of the catheter 402 such that the first opening 409 and the second opening 411 are fluidically isolated from the inflation lumen 448 of the valve 440. Accordingly, when the valve 440 is in the first position, the interior region 422 of the first expandable member 420 and the interior region 432 of the second expandable member 430 are fluidically isolated from each other and from the inflation lumen 448.

When the valve 440 is in the second position, the second port 444 of the valve 440 is aligned with the second opening 411 of the catheter 402 such that the interior region 432 of the second expandable member 430 is placed in fluid communication with the inflation lumen 448. The first port 442 of the valve 440 is circumferentially offset from the first opening 409 of the catheter 402 such that the interior region 422 of the first expandable member 420 is fluidically isolated from the inflation lumen 448. Said another way, when the valve 440 is in the second position, a portion of the outer surface 447 of the valve 440 is disposed about the first opening 409 of the catheter 402 such that the first opening 409 (and therefore the interior region 422 of the first expandable member 420) is fluidically isolated from the inflation lumen 448. In this manner, when the valve 440 is in the second position, the second expandable member 430 can be moved between its collapsed configuration and its expanded configuration independently from the movement of the first expandable member 420. Said another way, when the valve 440 is in the second position, the inflation lumen 448 is in fluid communication with the interior region 432 of the second expandable member 430 and the interior region 432 of the second expandable member 430 is fluidically isolated from the interior region 422 of the first expandable member 420. In some embodiments, for example, the second expandable member 430 can be expanded such that a portion of the second expandable member 430 contacts and/or engages a portion of the patient's body to maintain a location and/or orientation of the catheter 402 and/or the first expandable member 420 within the patient's body.

As shown in FIG. 13, when the valve 440 is in the third position, the first port 442 of the valve 440 is aligned with the first opening 409 of the catheter 402 such that the interior region 422 of the first expandable member 420 is in fluid communication with the inflation lumen 448. The second port 444 of the valve 440 is circumferentially offset from the second opening 411 of the catheter 402 such that the interior region 432 of the second expandable member 430 is fluidically isolated from the inflation lumen 448. Said another way, when the valve 440 is in the third position, a portion of the outer surface 447 of the valve 440 is disposed about the second opening 411 of the catheter 402 such that the second opening 411 (and therefore the interior region 432 of the second expandable member 430) is fluidically isolated from the inflation lumen 448. In this manner, when the valve 440 is in the third position, the first expandable member 420 can be moved between its collapsed configuration and its expanded configuration independently from the movement of the second expandable member 430. Said another way, when the valve 440 is in the third position, the inflation lumen 448 is in fluid communication with the interior region 422 of the first expandable member 420 and the interior region 422 of the first expandable member 420 is fluidically isolated from the interior region 432 of the second expandable member 430. In some embodiments, for example, the first expandable member 420 can be expanded such that a portion of the first expandable member 420 contacts and/or engages a portion of the patient's body to move, displace, compact and/or compress a bodily tissue (e.g., a bone, a cartilage or the like).

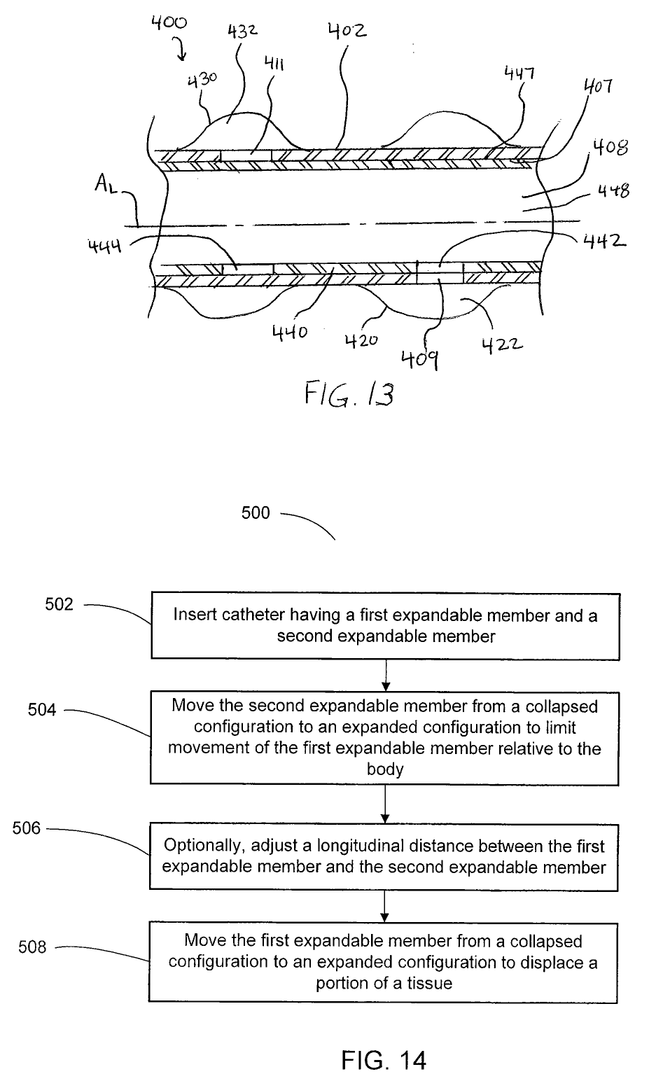

FIG. 14 is a flow chart of a method 500 of deploying expandable members within a body according to an embodiment of the invention. The method includes inserting into a body a catheter having a first expandable member and a second expandable member, 502. The catheter can be any catheter having at least a first and second expandable member of the types shown and described herein. For example, in some embodiments, the first expandable member and/or the second expandable member can be configured to displace a bone structure. In some embodiments, for example, the inserting can include inserting the catheter into a bone structure, such as a vertebral body.

In some embodiments, for example, the method can optionally include percutaneously defining a passageway within a body of a patient. For example, in some embodiments, an access passageway having any suitable size and/or shape can be defined by a trocar. A cannula can then be disposed within the passageway. The catheter can then be inserted into the patient's body via the cannula.

The second expandable member is moved from a collapsed configuration to an expanded configuration such that the second expandable member limits movement of the first expandable member relative to the body, 504. For example, in some embodiments, the second expandable member can be moved such that the second expandable member engages a structure within the body (e.g., a portion of the bone structure, a surface of a bodily tissue, or the like) when in its expanded configuration. In this manner, the second expandable member can “anchor” the first expandable member (e.g., maintain an orientation and/or a position of the first expandable member) within the body.

In some embodiments, the method optionally includes adjusting a longitudinal distance between the first expandable member and the second expandable member, 506. For example, as described herein, in some embodiments, the catheter can be configured to selectively move the first expandable member longitudinally relative to the second expandable member. In this manner, the first expandable member can be accurately positioned within the body. In some embodiments, for example, the first expandable member can be moved longitudinally relative to the second expandable member after the second expandable member has been moved to its expanded configuration.

The first expandable member is moved from a collapsed configuration to an expanded configuration such that the first expandable member displaces a portion of a tissue, 508. In some embodiments, for example, the first expandable member can move an endplate of a vertebra when the first expandable member is moved from its collapsed configuration to its expanded configuration. In other embodiments, the first expandable member can compress a cancellous bone portion of a vertebral body when moved from its collapsed configuration to its expanded configuration.

In some embodiments, the second expandable member is moved from its collapsed configuration to its expanded configuration before the first expandable member is moved from its collapsed configuration to its expanded configuration. Similarly, in some embodiments, the second expandable member is moved from its collapsed configuration to its expanded configuration independently from when the first expandable member is moved from its collapsed configuration to its expanded configuration.

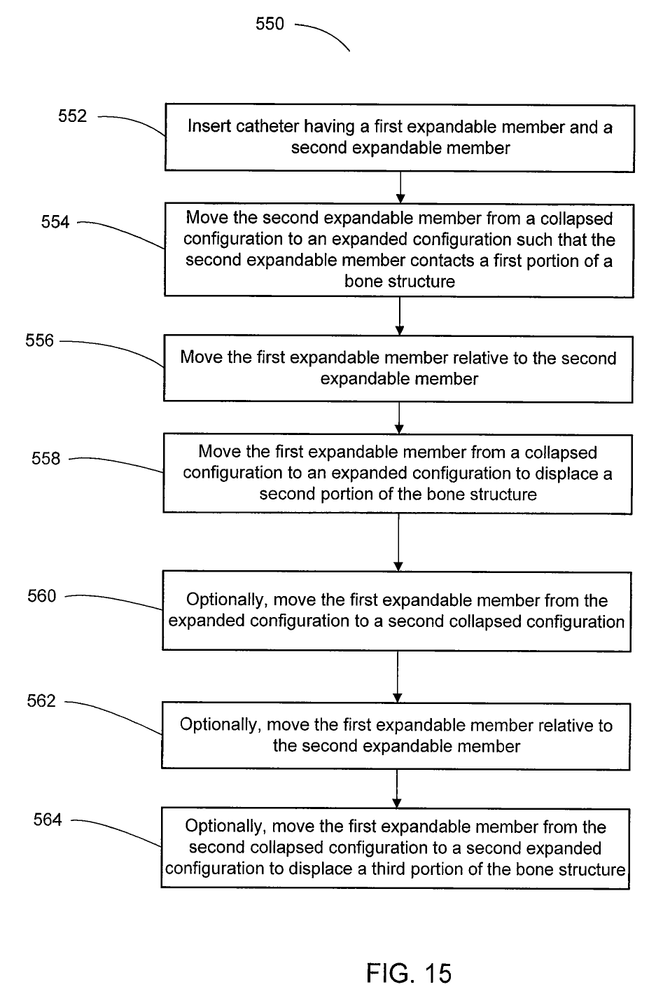

FIG. 15 is a flow chart of a method 550 of deploying expandable members within a body according to an embodiment of the invention. The method includes inserting into a body a catheter having a first expandable member and a second expandable member, 552. The catheter can be any catheter having at least a first and second expandable member of the types shown and described herein. For example, in some embodiments, the first expandable member and/or the second expandable member can be configured to displace a bone structure. In some embodiments, for example, the inserting can include inserting the catheter into a bone structure, such as a vertebral body.

The second expandable member is moved from a collapsed configuration to an expanded configuration such that the second expandable member contacts a first portion of a bone structure, 554. For example, in some embodiments, the second expandable member can be moved such that the second expandable member contacts a portion of an access passageway within a pedicle of a vertebra when in its expanded configuration. In this manner, the second expandable member can “anchor” the catheter within the body (e.g., the second expandable member can maintain an orientation and/or a position of the first expandable member within the body).

After the second expandable member is moved from its collapsed configuration to its expanded configuration, the first expandable member is moved relative to the second expandable member, 556. In this manner, the location and/or orientation of the first expandable member can be adjusted after the catheter is anchored within the body. In some embodiments, for example, the first expandable member can be moved along a longitudinal axis of the catheter relative to the second expandable member. In other embodiments, the first expandable member can be rotated about a longitudinal axis of the catheter relative to the second expandable member.

The first expandable member is moved from a collapsed configuration to an expanded configuration such that the first expandable member displaces a second portion of the bone structure, 558. In some embodiments, the second portion of the bone structure can be different than the first portion of the bone structure. For example, in some embodiments, the first expandable member can move an endplate of a vertebra when the first expandable member is moved from its collapsed configuration to its expanded configuration. In other embodiments, the first expandable member can compress a cancellous bone portion of a vertebral body when moved from its collapsed configuration to its expanded configuration.

In some embodiments, the second expandable member is moved from its collapsed configuration to its expanded configuration independently from when the first expandable member is moved from its collapsed configuration to its expanded configuration. For example, in some embodiments, a first inflation medium can be used to move the first expandable member from its collapsed configuration to its expanded configuration, and a second inflation medium different than the first inflation medium can be used to move the second expandable member from its collapsed configuration to its expanded configuration.

In some embodiments, the method can optionally include moving the first expandable member from the its expanded configuration to a second collapsed configuration, 560; moving the first expandable member relative to the second expandable member, 562; and moving the first expandable member from the second collapsed configuration to a second expanded configuration, 564. Said another way, in some embodiments, the first expandable member can be collapsed, repositioned within the body, and re-expanded. In this manner, the first expandable member can displace the second portion of the bone structure via a series of sequential steps between which the position of the first expandable member is changed. Moreover, in some embodiments the second portion of the bone structure and a third portion of the bone structure can be moved via the first expandable member.

In some embodiments, a second collapsed configuration can be different than a first collapsed configuration. For example, in some embodiments, the first collapsed configuration can be a fully collapsed configuration and the second collapsed configuration can be a partially collapsed configuration. Similarly, in some embodiments, the second expanded configuration can be different than the first expanded configuration. For example, in some embodiments, the first expandable member can have a first size, pressure and/or volume when in the first expanded configuration different from a size, pressure and/or volume when in the second expanded configuration.

In some embodiments, the first expandable member can be moved along a longitudinal axis of the catheter relative to the second expandable member after the first expandable member is in its second collapsed configuration. In other embodiments, the first expandable member can be rotated about a longitudinal axis of the catheter relative to the second expandable member after the first expandable member is in its second collapsed configuration. For example, in some embodiments, a first expandable member can have a circumferential shape that is asymmetrical about the longitudinal axis. In such embodiments, the first expandable member can be configured to be rotated about the longitudinal axis such that certain portions of the circumference of the first expandable member can be selectively positioned within the body.

The expandable members shown and described herein can be constructed from any material having suitable properties for being inserted percutaneously into a body, anchoring against a bodily structure and/or displacing a bodily tissue. For example, in some embodiments, the expandable members shown and described herein can be constructed from any material having suitable properties for being inserted percutaneously into a bone structure, anchoring against a bone structure, compacting bone material and/or displacing bone material. Such material properties can include, for example, biocompatibility, resistance to corrosion and/or degradation, high tensile strength, high tear resistance, high puncture resistance, high lubricity, suitable hardness, compliance (e.g., the expandable member's ability to expand appreciably beyond its nominal size) and/or elasticity. Moreover, the material properties suitable for operation within a bone structure can be different than the material properties that may be suitable for expandable members operating in other regions of a patient's body. Said another way, an expandable member suitable for use in the cardiovascular system may not be suitable for use in bone structures because of the nature of such bone structures, which can include multiple regions of bone having different densities, sharp protrusions, narrow access channels and the like, and because of the intended operation of the expandable member within the bone structure, which can include compacting bone and/or displacing bone.

In some embodiments, for example, a first expandable member (e.g. expandable members 120, 220, 320 or 420) and a second expandable member (e.g., expandable members 130, 230, 330 or 430) can be constructed from the same material and have substantially the same performance characteristics. In other embodiments, a first expandable member and a second expandable member can be constructed from the different materials and can have different performance characteristics. For example, in some embodiments, the first expandable member can be a low-compliant balloon configured to compact and/or displace bone material without the low-compliant balloon significantly deforming, and the second expandable member can be a high-compliant balloon configured to significantly elastically deform and engage a portion of a bone structure when the high-compliant balloon is expanded.

As used herein, the compliance of a balloon is the degree to which a size of the balloon in an unfolded state changes as a function of the pressure within the balloon. For example, in some embodiments, the compliance of a balloon can be used to characterize the change in the diameter of the unfolded balloon as a function of the balloon pressure. In some embodiments, for example, the diameter of an unfolded balloon characterized as a low-compliant balloon can change by zero to ten percent over the range of inflation pressure. In other embodiments, an unfolded balloon in which the diameter changes by as much as 20 percent may be characterized as a low-compliant balloon. Similarly, in some embodiments, the diameter of an unfolded balloon characterized as a high-compliant balloon can change by 18 to 30 percent. In other embodiments, the diameter of an unfolded high-compliant balloon can change by as much as 100 to 600 percent over the range of inflation.

In some embodiments, the compliance of a balloon can be used to characterize the change in the length of the balloon as a function of the balloon pressure. The change in length can also be referred to as the elongation percentage of the balloon. In other embodiments, the compliance of a balloon can be used to characterized the change in volume of the balloon as a function of the balloon pressure. Similarly, in some embodiments, the compliance of a balloon can be used to characterize the material properties from which the balloon or portions of the balloon are constructed.

In some embodiments, for example, an expandable member can be constructed from a low-compliant material (e.g., a material having a low modulus of elasticity), such as polyamide, polyethylene terephthalate (PET), Nylons, cross-linked Polyethylene, PEBAX®, Polyurethanes, PVC or any blend of these compounds. In some embodiments, an expandable member can be constructed from Nylon 12.

In some embodiments, for example, an expandable member can be constructed from a material configured to allow a therapeutic agent to pass therethrough. For example, in some embodiments, an expandable member can be constructed from a porous material configured to selectively allow a drug to pass from an interior region of the expandable member to an area outside of the expandable member (i.e., within the body). In this manner, an expandable member can delivery a therapeutic agent to a predetermined region within the body.

While various embodiments of the invention have been described above, it should be understood that they have been presented by way of example only, and not limitation. Where methods described above indicate certain events occurring in certain order, the ordering of certain events may be modified. Additionally, certain of the events may be performed concurrently in parallel when possible, as well as performed sequentially as described above. While specific embodiments have been particularly shown and described, it will be understood that various changes in form and details may be made.

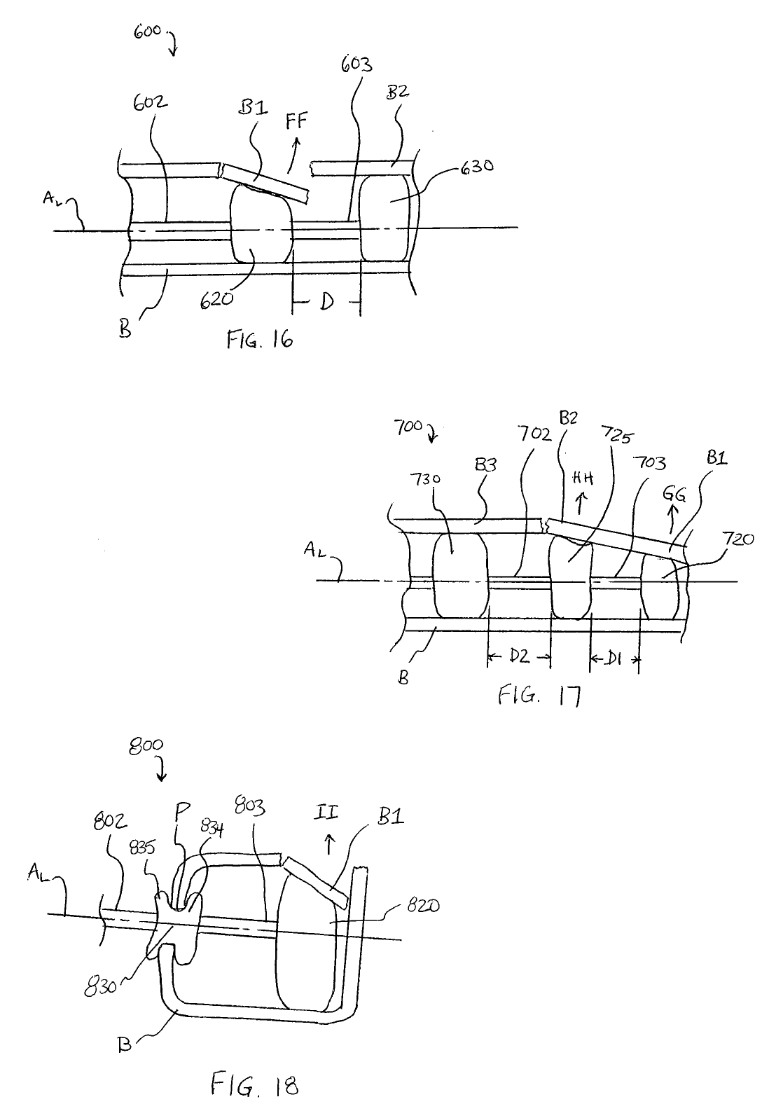

For example, although the medical device 100 is shown and described as having a catheter 102 having a first expandable member 120 spaced distally from a second expandable member 130, in some embodiments, a catheter can include a first expandable member disposed proximally from a second expandable member. Said another way, in some embodiments, an anchoring balloon can be disposed distally relative to a tissue displacement balloon. For example, FIG. 16 is a schematic illustration of a medical device 600 according to an embodiment of the invention disposed within a bone structure B. The medical device 600 includes a catheter 602, a first expandable member 620 and a second expandable member 630.

The first expandable member 620 is coupled to the catheter 602 at a first position along the longitudinal axis A_{L} of the catheter 602. The second expandable member 630 is coupled to a distal end 603 of the catheter 602 at a second position along the longitudinal axis A_{L} of the catheter 602 such that the first expandable member 620 and the second expandable member 630 are spaced apart by a distance D. In this manner, the second expandable member 630 can be positioned adjacent a second portion B2 of the bone structure B, and the first expandable member 620 can be positioned adjacent a first portion B1 of the bone structure B.

As described above, the second (or anchoring) expandable member 630 has a collapsed configuration (not shown in FIG. 16) and an expanded configuration (as shown in FIG. 16). When the second expandable member 630 is in its expanded configuration, a portion of the second expandable member 630 contacts and/or engages the second portion B2 of the bone structure B. In this manner, the second expandable member 630 can anchor the medical device 600 within a distal portion of the bone structure B. In some embodiments, for example, the distal portion of the bone structure B may provide an advantageous location for anchoring the medical device 600.

The first (or tissue displacement) expandable member 620 also has a collapsed configuration and an expanded configuration. As shown in FIG. 16, when the first expandable member 620 moves from its collapsed configuration to its expanded configuration, a portion of the first expandable member 620 contacts and/or engages the first portion B1 of the bone structure B to move, compress and/or compact the first portion B1 as indicated by the arrow FF.

Although the medical device 600 is shown and described as including a second expandable member 630 configured to maintain a location, position and/or orientation of a first expandable member 620 within a patient's body, in some embodiments, a medical device can include multiple expandable members configured to maintain a location, position and/or orientation of a first expandable member within a patient's body. For example, in some embodiments, a medical device can include a first anchoring member disposed distally from an expandable member and a second anchoring member disposed proximally from the expandable member, the first anchoring member and the second anchoring member being configured to collectively maintain a location, position and/or orientation of the expandable member within a patient's body. In other embodiments, a medical device can include multiple anchoring members, each configured to individually maintain a location, position and/or orientation of an expandable member within a patient's body.

Similarly, although the medical device 600 is shown and described as including a second expandable member 630 disposed at the distal end 603 of the catheter 602, in other embodiments, a medical device can include a second expandable member disposed at a distal end portion of a catheter. For example, in some embodiments, catheter can include a trocar tip, sensor or the like at the distal end of the catheter, and can include an expandable member at the distal end portion of the catheter (i.e., disposed proximally from the trocar tip, sensor or the like). In other embodiments, a catheter can be configured to receive a guide wire such that the guide wire extends from the distal end of the catheter. In such embodiments, the catheter can include an expandable member at the distal end portion of the catheter.

Although the medical device 600 is shown and described as including a first expandable member 620 configured to move, compress and/or compact a bodily tissue (e.g., a bone structure), in some embodiments, a medical device can include multiple expandable members configured to move, compress and/or compact a bodily tissue. For example, FIG. 17 is a schematic illustration of a medical device 700 according to an embodiment of the invention disposed within a bone structure B. The medical device 700 includes a catheter 702, a first expandable member 720, a second expandable member 725, and a third expandable member 730.

The first expandable member 720 is coupled to a distal end 703 of the catheter 702 at a first position along the longitudinal axis A_{L} of the catheter 702. The second expandable member 725 is coupled to the catheter 702 at a second position along the longitudinal axis A_{L} of the catheter 702 such that the first expandable member 720 and the second expandable member 725 are spaced apart by a first distance D1. The third expandable member 730 is coupled to the catheter 702 at a third position along the longitudinal axis A_{L} of the catheter 702 such that the second expandable member 725 and the third expandable member 730 are spaced apart by a second distance D2. In this manner, the first expandable member 720 can be positioned adjacent a first portion B1 of the bone structure B, the second expandable member 725 can be positioned adjacent a second portion B2 of the bone structure B, and the third expandable member 730 can be positioned adjacent a third portion B3 of the bone structure B.

As described above, the third (or anchoring) expandable member 730 has a collapsed configuration (not shown in FIG. 17) and an expanded configuration (as shown in FIG. 17). When the third expandable member 730 is in its expanded configuration, a portion of the third expandable member 730 contacts and/or engages the third portion B3 of the bone structure B. In this manner, the third expandable member 730 can anchor the medical device 700 within the bone structure B. Said another way, when the third expandable member 730 is in its expanded configuration the third expandable member 730 contacts and/or engages the third portion B3 of the bone structure to maintain a location, position and/or orientation of the first expandable member 720 and/or the second expandable member 725 within the bone structure B.

The first expandable member 720 and the second expandable member 725 each have a collapsed configuration and an expanded configuration. As shown in FIG. 17, when the first expandable member 720 moves from its collapsed configuration to its expanded configuration, a portion of the first expandable member 720 contacts and/or engages the first portion B1 of the bone structure B to move, compress and/or compact the first portion B1 as indicated by the arrow GG. Similarly, when the second expandable member 725 moves from its collapsed configuration to its expanded configuration, a portion of the second expandable member 725 contacts and/or engages the second portion B2 of the bone structure B to move, compress and/or compact the second portion B2 as indicated by the arrow HH. In some embodiments, the first expandable member 720 and the second expandable member 725 can be moved from their respective collapsed configurations to their respective expanded configurations substantially simultaneously. In other embodiments, the first expandable member 720 and the second expandable member 725 can be moved from their respective collapsed configurations to their respective expanded configurations sequentially.

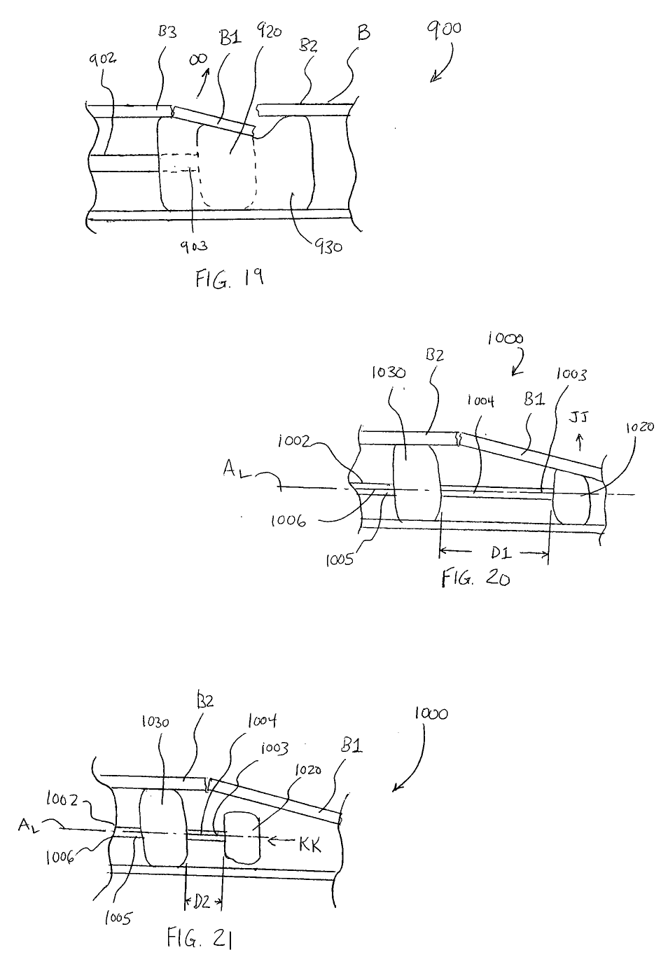

Although the anchoring expandable member 730 is shown as being disposed within the bone structure B and engaging an interior portion of the third portion B3 of the bone structure, in other embodiments, an anchoring expandable member can contact and/or engage any portion of a bodily structure to maintain a position, location and/or orientation of a displacement expandable member. For example, FIG. 18 is a schematic illustration of a medical device 800 according to an embodiment of the invention disposed within a bodily structure B, such as for example a vertebral body. The medical device 800 includes a catheter 802, a first expandable member 820 disposed at a distal end 803 of the catheter, and a second expandable member 830.

The second (or anchoring) expandable member 830 has a collapsed configuration (not shown in FIG. 18) and an expanded configuration (as shown in FIG. 18). In use, the second (or anchoring) expandable member 830 can be disposed within a passageway P defined within the bodily structure B. When in its expanded configuration, the second expandable member 830 is shaped such that a first portion 834 of the second expandable member 830 is disposed within the bodily structure B and a second portion 835 of the second expandable member 830 is disposed outside of the bodily structure B. Similarly stated, when in its expanded configuration, the second expandable member 830 is shaped to matingly fit within the passageway P. In this manner, the second expandable member 830 can maintain a position, location and/or orientation of the first expandable member 820 within the bodily structure B.

The first (or tissue displacement) expandable member 820 has a collapsed configuration and an expanded configuration. As shown in FIG. 18, when the first expandable member 820 moves from its collapsed configuration to its expanded configuration, a portion of the first expandable member 820 contacts and/or engages the first portion B1 of the bone structure B to move, compress and/or compact the first portion B1 as indicated by the arrow II.

Although the second expandable member 830 is shown in FIG. 18 as having an “H-like” cross-sectional shape, in other embodiments, the second expandable member 830 can have any suitable shape to matingly fit within and/or to correspond to a bodily structure to which the medical device 800 is being anchored. For example, in some embodiments, an expandable member can be shaped to fit within an opening defined by a bone structure when the expandable member is in an expanded configuration. In other embodiments, an expandable member can be shaped to fit within and/or correspond to a bodily structure other than bone.

Although the medical devices shown and described above include a first expandable member (e.g., a displacement expandable member) spaced apart from a second expandable member (e.g., an anchoring expandable member), in some embodiments, a medical device can include a first expandable member having a portion configured to contact a portion of a second expandable member. For example, in some embodiments, a catheter can include a first expandable member disposed adjacent a second expandable member longitudinally such that a side portion of the first expandable member contacts a side portion of the second expandable member when the first expandable member and/or the second expandable member are in an expanded configuration. In other embodiments, a catheter can include a first expandable member and a second expandable member disposed at the same longitudinal position, but with the first expandable member disposed adjacent the second expandable member circumferentially such that a side portion of the first expandable member contacts a side portion of the second expandable member when the first expandable member and/or the second expandable member are in an expanded configuration. In yet other embodiments, a catheter can include a first expandable member disposed within a second expandable member such that a portion of the first expandable member contacts a portion of the second expandable member. For example, FIG. 19 is a schematic illustration of a medical device 900 according to an embodiment of the invention disposed within a bone structure B. The medical device 900 includes a catheter 902, a first expandable member 920 and a second expandable member 930.

The first expandable member 920 is coupled to a distal end 903 of the catheter 902. The second expandable member 930 is coupled to the distal end 903 of the catheter 902 such that the first expandable member 920 is disposed within the second expandable member 930. In this manner, the first expandable member 920 can be positioned adjacent a first portion B1 of the bone structure B, and the second expandable member 930 can be positioned adjacent the first portion B1, a second portion B2, and a third portion B3 of the bone structure B. Similarly stated, the first expandable member 920 and the second expandable member 930 can be collectively positioned adjacent the first portion B1 of the bone structure B, where the second expandable member 930 has portions also adjacent the second portion B2 and the third portion B3 of the bone structure B.

As described above, the second (or anchoring) expandable member 930 has a collapsed configuration (not shown in FIG. 19) and an expanded configuration (as shown in FIG. 19). When the second expandable member 930 is in its expanded configuration, a portion of the second expandable member 930 contacts and/or engages the second portion B2 and/or the third portion B3 of the bone structure B. In this manner, the second expandable member 930 can anchor the medical device 900 the bone structure B.

The first (or tissue displacement) expandable member 920 also has a collapsed configuration and an expanded configuration. As shown in FIG. 19, when the first expandable member 920 moves from its collapsed configuration to its expanded configuration, a portion of the first expandable member 920 contacts and/or engages the first portion B1 of the bone structure B to move, compress and/or compact the first portion B1 as indicated by the arrow OO.

Although shown as contacting and/or engaging the first portion B1 of the bone structure B, in some embodiments, the second expandable member 930 is not configured to move, displace, compact and/or compress the first portion B1 of the bone structure B. For example, in some embodiments, the second expandable member 930 can be constructed from a high compliant material suitable for conforming to portions of a bone for anchoring, but not suitable for moving, displacing, compacting and/or compressing bone.

In other embodiments, however, the second expandable member 930 can be configured to move, displace, compact and/or compress the first portion B1 of the bone structure B. For example, in some embodiments, the second expandable member 930 can be configured to move the first portion B1 of the bone structure B by a first distance and the first expandable member 920 can be configured to move the first portion B1 of the bone structure B by a second distance. Said another way, in some embodiments, the first expandable member 920 and the second expandable member 930 can cooperatively move, displace, compact and/or compress the first portion B1 of the bone structure B.

Although the medical devices are shown and described above as having a catheter having a first expandable member spaced apart from a second expandable member by a fixed distance, in other embodiments a first expandable member can be configured to move relative to a second expandable member. In some embodiments, for example, a catheter having a first expandable member and a second expandable member can be configured to selectively adjust a longitudinal distance between the first expandable member and the second expandable member. For example, FIGS. 20-22 are schematic illustrations of a medical device 1000 according to an embodiment of the invention disposed within a bone structure B in a first configuration, a second configuration and a third configuration, respectively.

The medical device 1000 includes a catheter 1002, a first expandable member 1020 and a second expandable member 1030. The catheter has a first shaft 1004 and a second shaft 1006. As described in more detail herein, the first shaft 1004 is movable relative to the second shaft 1006. The first expandable member 1020 is coupled to a distal end of the first shaft 1004 of the catheter 1002. The second expandable member 1030 is coupled to a distal end 1005 of the second shaft 1006 of the catheter 1002. As described in more detail herein, the first expandable member 1020 is spaced apart from the second expandable member 1030 by a selectively adjustable distance along the longitudinal axis A_{L} of the catheter 1002. In this manner, the second expandable member 1030 can be positioned adjacent a second portion B2 of the bone structure B, and the first expandable member 1020 can be positioned adjacent a first portion B1 of the bone structure B.

As described above, the second expandable member 1030 has a collapsed configuration (not shown in FIGS. 20-22) and an expanded configuration (as shown in FIGS. 20-22). When the second expandable member 1030 is in its expanded configuration, a portion of the second expandable member 1030 contacts and/or engages the second portion B2 of the bone structure B. In this manner, the second expandable member 1030 can anchor the medical device 1000 within the bone structure B. The first expandable member 1020 also has a collapsed configuration and an expanded configuration.

As shown in FIG. 20, when the medical device 1000 is in the first configuration, the first expandable member 1020 is spaced apart from the second expandable member 1030 by a distance D1 along the longitudinal axis A_{L} of the catheter 1002. In this manner, the first expandable member 1020 can be positioned adjacent the distal portion of the first portion B1 of the bone structure B. Accordingly, when the first expandable member 1020 moves from its collapsed configuration to its expanded configuration, a portion of the first expandable member 1020 contacts and/or engages the distal portion of the first portion B1 of the bone structure B to move, compress and/or compact the first portion B1 as indicated by the arrow JJ in FIG. 20.

As shown in FIG. 21, the first expandable member 1030 can be moved from its expanded configuration to a second collapsed configuration different than the initial collapsed configuration. Once the first expandable member 1030 is in its second collapsed configuration, the first shaft 1004 of the catheter 1002 can be moved relative to the second shaft 1006 of the catheter 1002 to selectively adjust the longitudinal distance between the first expandable member 1020 relative to the second expandable member 1030, as indicated by the arrow KK. Said another way, when the medical device 1000 is in the second configuration, the first expandable member 1020 is spaced apart from the second expandable member 1030 by a distance D2 along the longitudinal axis A_{L} of the catheter 1002. In this manner, the first expandable member 1020 can be positioned adjacent the proximal portion of the first portion B1 of the bone structure B. Although the distance D2 is shown as being less than the distance D1, in other embodiments, the distance D2 can be greater than the distance D1.

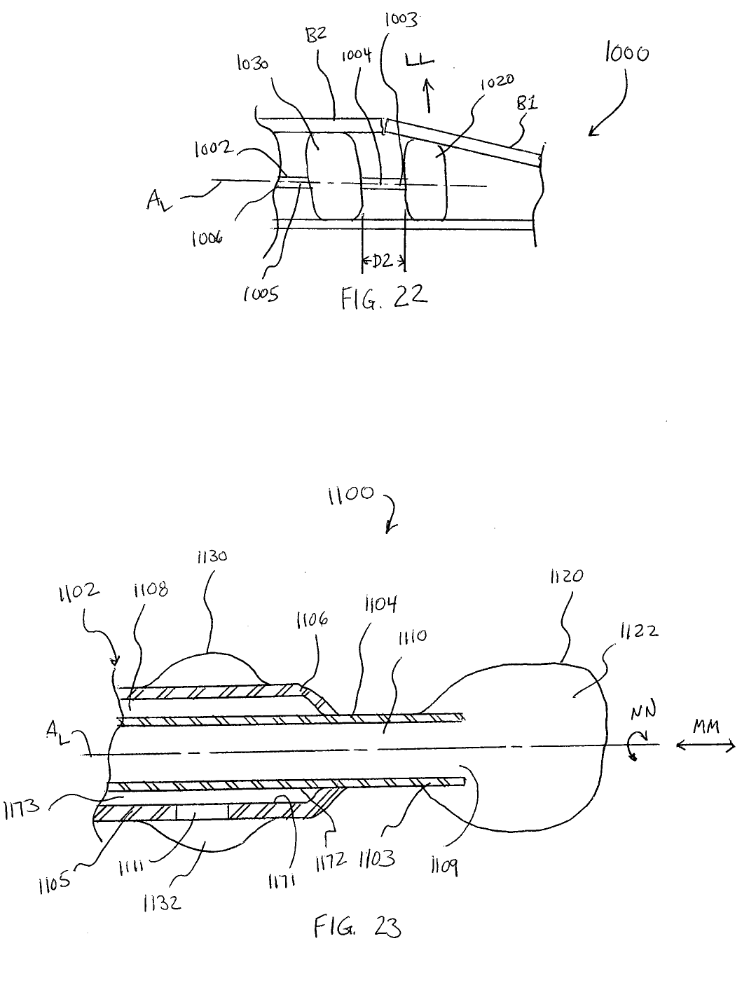

When the first expandable member 1020 is spaced apart from the second expandable member 1030 by the distance D2, the first expandable member can be moved from it second collapsed configuration to a second expanded configuration, as shown in FIG. 22. When the first expandable member 1020 moves from its second collapsed configuration to its second expanded configuration, a portion of the first expandable member 1020 contacts and/or engages the proximal portion of the first portion B1 of the bone structure B to move, compress and/or compact the first portion B1 of the bone structure B as indicated by the arrow LL in FIG. 22. In this manner, the first expandable member 1020 can sequentially apply a force to predetermined portions of the first portion B1 of the bone structure B.

FIG. 23 is a partial cross-sectional view of a medical device 1100 according to an embodiment of the invention. The medical device 1100 includes a catheter 1102, a first expandable member 1120 and a second expandable member 1130. The catheter has a first shaft 1104 movably disposed within a second shaft 1106. The first expandable member 1120 is coupled to a distal end 1103 of the first shaft 1104 and defines an interior region 1122 within which an inflation medium can be conveyed to move the first expandable member 1120 from its collapsed configuration to its expanded configuration. The second expandable member 1130 is coupled to a distal end 1105 of the second shaft 1106 and defines a interior region 1132 within which an inflation medium can be conveyed to move the second expandable member 1130 from its collapsed configuration to its expanded configuration.

The first shaft 1104 of the catheter 1102 defines an inflation lumen 1110 and an opening 1109 at the distal end of the first shaft 1104. The inflation lumen 1110 of the first shaft 1104 is in fluid communication with the interior region 1122 of the first expandable member 1120 via the opening 1109. Similarly, the second shaft 1106 of the catheter 1102 defines lumen 1108 and an opening 1111 in the side wall of the second shaft 1106. A portion of the first shaft 1104 is movably disposed within the lumen 1108 of the second shaft 1106 such that the inner surface 1171 of the second shaft 1106 and the outer surface 1172 of the first shaft 1104 define an inflation passageway 1173. The inflation passageway 1173 is in fluid communication with the interior region 1132 of the first expandable member 1130 via the opening 1111. In this manner, as described above, the first expandable member 1120 and the second expandable member 1130 can be moved between their respective collapsed and expanded configurations independently from each other. Moreover, this arrangement allows the first expandable member 1120 to be moved longitudinally and/or rotationally relative to the second expandable member 1130, as shown by the arrows MM and NN, respectively.

Although the catheter 1102 is shown and described as including a first shaft 1104 and a second shaft 1106, in some embodiments a catheter assembly can include any number of shafts, tubes and/or other components. For example, in some embodiments, a catheter assembly can include an outer shaft, an inner shaft and a stylet disposed within the inner shaft, similar to the catheter disclosed in U.S. Pat. No. 6,719,773, which is incorporated herein by reference in its entirety. In other embodiments, a catheter assembly can include an actuator configured to move the first shaft relative to the second shaft by a predetermined distance, similar to the actuators shown and described in U.S. patent application Ser. No. 11/730,349, entitled “Apparatus and Methods for Use of Expandable Members in Surgical Applications,” filed Mar. 30, 2007, which is incorporated herein by reference in its entirety.

Similarly, although the catheters shown and described herein are linear, in some embodiments, a catheter can include a curved portion. For example, in some embodiments a catheter can include a curved portion configured to be disposed within a portion of body. In other embodiments, a catheter can include a flexible portion configured to bend and/or twist within the body. For example, in some embodiments a medical device can include a steerable catheter.

Although the valve 440 is shown and described with reference to FIGS. 11-13 as rotating within the catheter 402, in other embodiments, a valve can instead translate within a catheter. In other embodiments, a valve can translate and rotate within a catheter. Similarly, although the valve 440 is shown and described above as having three positions, in some embodiments, a valve can have any number of positions. For example, in some embodiments, a valve can have a fourth position in which both a first expandable member and a second expandable member are simultaneously placed in fluid communication with an inflation lumen.

Although the first expandable members 120 and 220 are shown and described above as displacing a portion of a bone structure by moving, displacing and/or compacting that portion of the bone structure, in other embodiments, an expandable member can displace a spine-related tissue by compacting and/or moving the spine-related tissue. Such spine-related tissue can include, for example, portions of a vertebral body, ligaments of the spinal column, the annulus fibrosis of a disc, the nucleus pulposus of a disc, an implanted disc replacement material or the like.

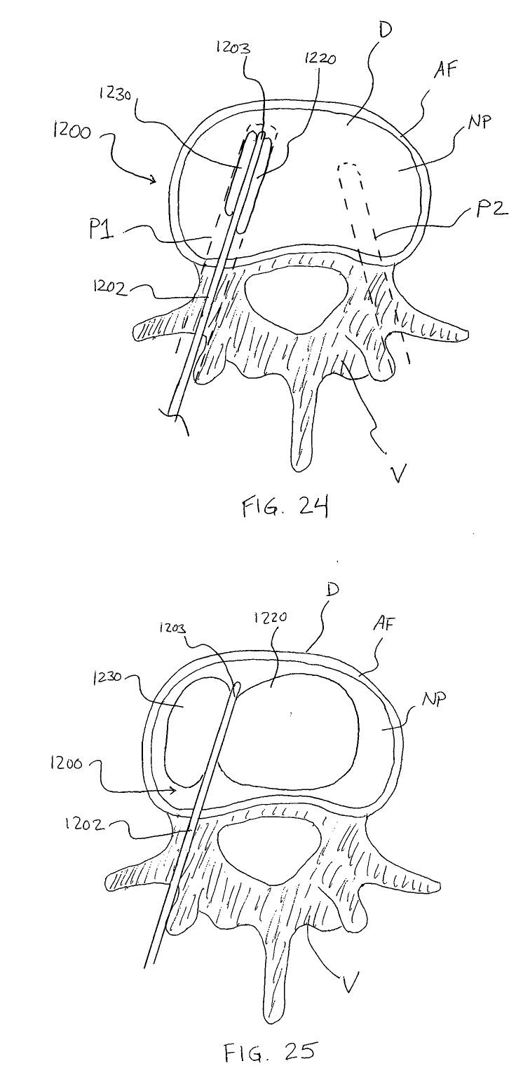

For example, FIGS. 24-28 are cross-sectional side views of a medical device 1200 from a transverse plane, according to an embodiment of the invention disposed within a disc D of a spinal column. The disc D is disposed between a pair of adjacent vertebrae (a portion of the inferior vertebra V is shown in FIGS. 24-28). The disc D includes an annulus fibrosis AF and region of nucleus pulposus NP disposed within the annulus fibrosis AF. The medical device 1200 includes a catheter 1202, a first expandable member 1220 disposed at a distal end 1203 of the catheter 1202 at a first circumferential position (i.e., the contralateral position), and a second expandable member 1230 disposed at the distal end 1203 of the catheter 1202 at a second circumferential position (i.e., the ipsilateral position).

As shown in FIG. 24, a first access passageway P1 can be defined to allow the medical device 1200 to be inserted into the disc D. The first passageway P1 can have any suitable shape (e.g., linear, curved in a single plane, curved in multiple planes or the like) and can be formed by any suitable tool, such as, for example, a trocar (not shown in FIGS. 24-28). Similarly, a second access passageway P2 can be defined through which a therapeutic material and/or device can be implanted into the disc D.

The medical device 1200 can be inserted into the body such that the second expandable member 1230 is disposed in the ipsilateral position within the disc D and the first expandable member 1220 is disposed in the contralateral position within disc D. As shown in FIG. 25, after the medical device 1200 is positioned within the disc D, the first expandable member 1220 and the second expandable member 1230 can be moved from their respective collapsed configurations to their respective expanded configurations. In this manner, at least the first expandable member 1220 can displace a portion of the nucleus pulposus NP and/or distract the adjacent vertebrae (only the inferior vertebra V is shown in FIGS. 24-28). Said another way, in this manner, at least the first expandable member 1220 can prepare the disc D for the implantation of a disc replacement material, a fusion cage, and/or the like.

As shown in FIG. 26, after the disc D is prepared for the implantation of a disc replacement material, a fusion cage, and/or the like, the first expandable member 1220 is then moved from its expanded configuration to its collapsed configuration. The second expandable member 1230 is maintained in its expanded configuration to maintain the space defined by the first expandable member 1220. Said another way, the second expandable member 1230 maintains the distraction of the adjacent vertebrae when the first expandable member 1220 is moved to its collapsed configuration. A disc replacement material 1261 and/or a fusion cage 1263 can then be implanted into the disc D by a medical device 1260 via the second passageway P2. The medical device 1260 can be any suitable device for implanting the disc replacement material 1261 and/or the fusion cage 1263. The disc replacement material 1261 can include any suitable disc replacement material or combination of materials, such as, for example, a hydrogel, a bone morphogenic protein (BMP), hydoxyapatite, tricalcium phosphate, demineralized bone matrix, Autograft, transforming growth factors, fibroblast growth factors, platelet derived growth factors, bone marrow aspirant, collagen matrices doped with graft material, or the like. Similarly, the fusion cage 1263 can be any suitable fusion cage, such as for example, a static fusion cage, an expandable fusion cage, and/or the like. The disc replacement material 1261 and/or the fusion cage 1263 can be implanted either after the first expandable member 1220 has been moved to its collapsed configuration or when the first expandable member 1220 is being moved between its expanded configuration and its collapsed configuration.

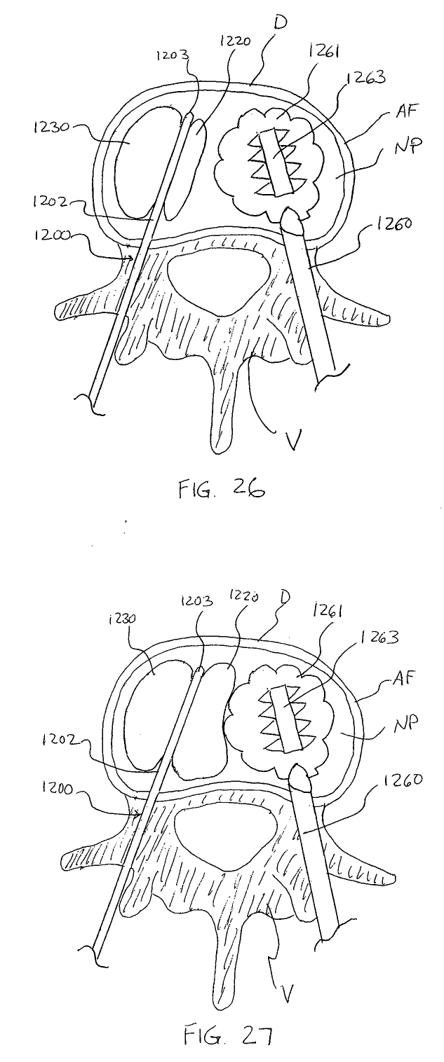

As shown in FIG. 27, in some embodiments, the first expandable member 1220 can be moved from its collapsed configuration to its expanded configuration (or a partially expanded configuration) after the implantation of the disc replacement material 1261 and/or the fusion cage 1263. Said another way, after the implantation of the disc replacement material 1261 and/or the fusion cage 1263, the first expandable member 1220 can be reexpanded to compact or “pack” the implanted disc replacement material 1261 within the disc D and/or the fusion cage 1263.

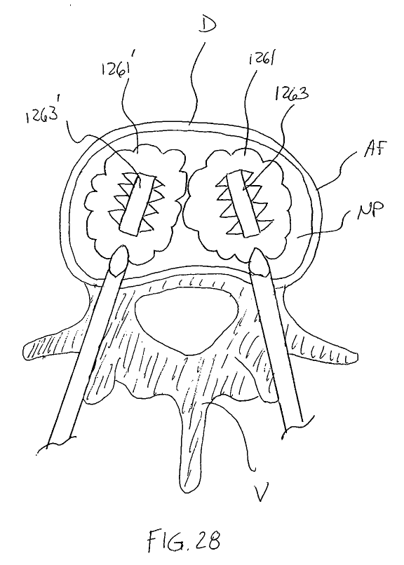

In some embodiments, the medical device 1200 can be used to facilitate a bilateral cage placement within the disc D. For example, as shown in FIG. 28, after the disc replacement material 1261 and/or the fusion cage 1263 have been implanted on the contralateral side, as described above, the second expandable member 1230 can be moved from its expanded configuration to its collapsed configuration. A second disc replacement material 1261′ and/or a second fusion cage 1263′ can then be implanted into the disc D via the first passageway P1. In this manner, the medical device 1200 can be used to symmetrically distract adjacent vertebrae in preparation for the implantation of fusion cages and/or disc replacement materials for a bilateral fusion.

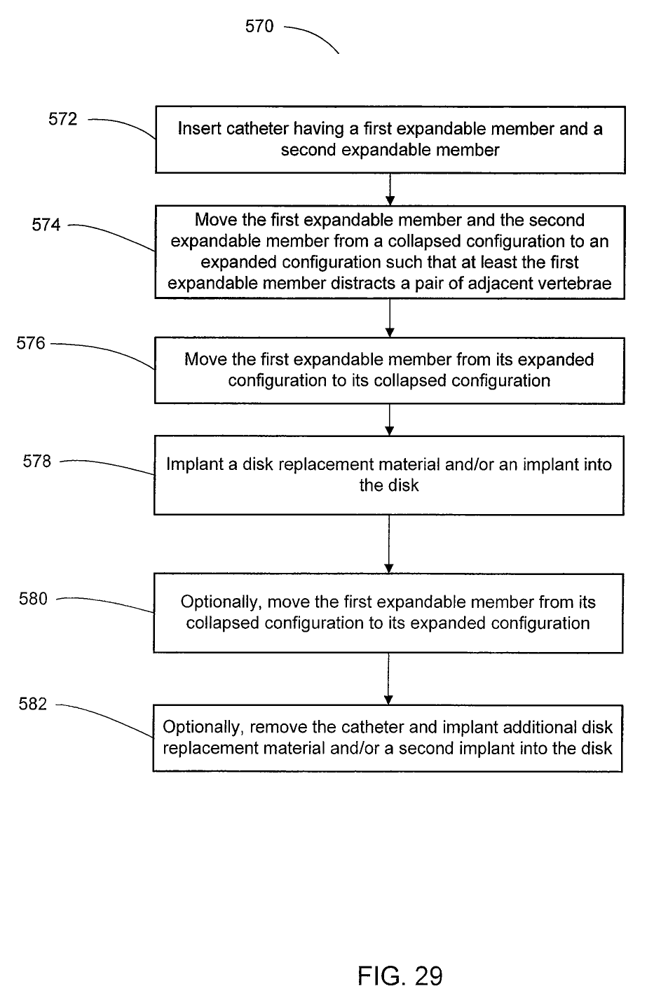

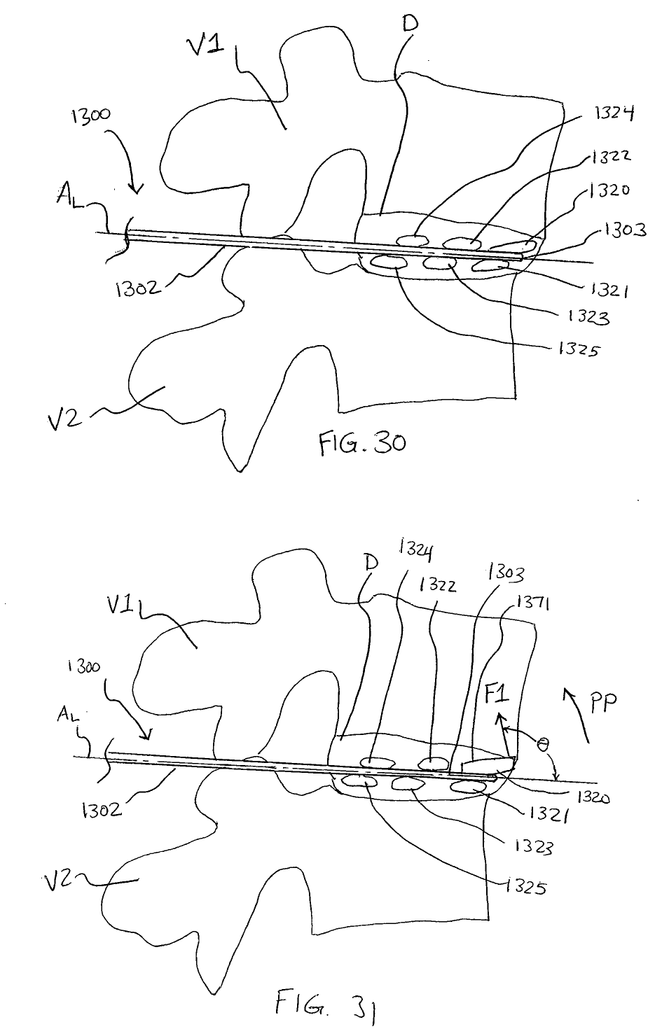

FIG. 29 is a flow chart of a method 570 of deploying expandable members within a disc of a spinal column according to an embodiment of the invention. The method includes inserting into the disc a catheter having a first expandable member and a second expandable member, 572. The catheter can be any catheter having at least first and second expandable members of the types shown and described herein. For example, in some embodiments, the first expandable member and/or the second expandable member can be disposed at a distal end of the catheter at different circumferential positions, similar to the catheter as shown in FIGS. 24-28.

The first expandable member and the second expandable member are then moved from their respective collapsed configurations to their respective expanded configurations such that at least the first expandable member distracts a pair of adjacent vertebrae, 574. In some embodiments, the first expandable member can displace and/or compress a portion of the nucleus pulposus within the disc when the first expandable member is moved from its collapsed configuration to its expanded configuration. In this manner, the first expandable member can define an implantation region within the disc. In some embodiments, the second expandable member can be disposed within the disc such that the second expandable member engages a structure within the body (e.g., a portion of the annulus fibrosis of the disc) when in its expanded configuration. In this manner, the second expandable member can “anchor” the first expandable member (e.g., maintain an orientation and/or a position of the first expandable member) within the body.

The first expandable member is moved from its expanded configuration to its collapsed configuration, 576. At least one of a disc replacement material or an implant is then implanted into the implantation region defined by the first expandable member, 578. In some embodiments, for example, the disc replacement material can be a hydrogel, a bone morphogenic protein (BMP), human hair, or the like. Similarly, in some embodiments, the implant can be a fusion cage, an expandable implant, and/or the like.

In some embodiments, the method optionally includes moving the first expandable member from its collapsed configuration to its expanded configuration after the disc replacement material and/or the implant has been implanted into the disc, 580. In this manner, the disc replacement material and/or the implant can be “packed” into or compacted within the implantation region defined by the first expandable member. In some embodiments, the first expandable member can be iteratively moved between its collapsed configuration and its expanded configuration. In some embodiments, additional disc replacement material can be implanted into the disc between iterations of the first expandable member. In some embodiments, the method optionally includes removing the catheter and implanting additional disc replacement material or a second implant, 582.

Although the medical device 700 is shown and described as including two expandable members that, in some embodiments, can be expanded sequentially to displace a portion of a bone structure, in other embodiments, a medical device can include any number of expandable members arranged in any orientation. Moreover, in some embodiments, the expandable members can be shaped to matingly fit within a portion of a patient's body in a predetermined orientation. For example, FIGS. 30 and 31 are schematic illustrations of a medical device 1300 according to an embodiment of the invention disposed within a disc D disposed between a first vertebra V1 (i.e., the superior vertebra) and a second vertebra V2 (i.e., the inferior vertebra). The medical device 1300 includes a catheter 1302, a first expandable member 1320, a second expandable member 1321, a third expandable member 1322, a fourth expandable member 1323, a fifth expandable member 1324, and a sixth expandable member 1325.

The first expandable member 1320 is coupled to a distal end 1303 of the catheter 1302 at a distal-most position along the longitudinal axis A_{L} of the catheter 1300. The second through sixth expandable members 1321-1325 are coupled to the distal end 1303 of the catheter 1302 at various longitudinal positions and various circumferential positions relative to each other and/or the first expandable member 1320. In this manner, as described below, when the expandable members 1320-1325 are in their respective expanded configurations, any number of different forces can be applied to the first vertebra V1 and/or the second vertebra V2.

As shown in FIG. 31, the first expandable member 1320 is shaped such that when the first expandable member 1320 is moved from its collapsed configuration to its expanded configuration a force F1 having a predetermined direction and/or magnitude is applied to the first vertebra V1 by the first expandable member 1320. More particularly, a first surface 1371 of the first expandable member 1320 is angularly offset from the longitudinal axis A_{L} of the catheter 1300 such that the resulting force F1 applied to the first vertebra V1 is offset from the longitudinal axis A_{L} by an angle Θ that is not orthogonal to the longitudinal axis A_{L}. Similarly, the second through sixth expandable members 1321-1325 can each be shaped such that they each produce a force on the first vertebra V1 and/or the second vertebra V2 having a predetermined direction and/or magnitude when in their respective expanded configurations (not shown in FIG. 31). In this manner, the expandable members 1320-1325 can be expanded in any number of combinations and/or sequences to produce a predetermined force or series of forces on the first vertebra V1 and/or the second vertebra V2. For example, in some embodiments, the medical device 1300 can be configured to produce a cantilever-type force on the first vertebra V1 to urge the first vertebra V1 in a direction as shown by the arrow PP in FIG. 31. Said another way, the medical device 1300 can be configured to produce a force having an upward and posterior direction on the first vertebra V1, which can both distract the vertebrae V1 and V2 and align the first vertebra V1 with second vertebra V2 to address misalignment of the spinal column (e.g., spondylolisthesis).

Although FIGS. 4-7 show the medical device 100 disposed within a vertebral body from an extrapedicular approach, in other embodiments, a medical device can be disposed within a vertebral body using any other suitable approach. For example, in some embodiments, a medical device can be disposed within a vertebral body using a transpedicular approach. In yet other embodiments, a medical device can be disposed within a vertebral body using an anterior approach.

Although various embodiments have been described as having particular features and/or combinations of components, other embodiments are possible having a combination of any features and/or components from any of embodiments where appropriate. For example, in some embodiments, a medical device can include a catheter, a first expandable member and a second expandable member. The catheter can have a first shaft movably disposed within a second shaft, similar to the catheter 1102 shown and described above. The catheter can also include a valve configured to selectively place an inflation lumen in fluid communication the first expandable member and the second expandable member while maintaining fluid isolation between the first expandable member and the second expandable member, similar to the valve 440 shown and described above.

What is claimed is:

1. An apparatus comprising:
a first expandable member having a collapsed configuration and an expanded configuration, the first expandable member configured to displace a portion of a bone when moved from its collapsed configuration to its expanded configuration; and
a second expandable member having a collapsed configuration and an expanded configuration, the second expandable member configured to limit movement of the first expandable member within a body of a patient when in its expanded configuration.

2. The apparatus of claim 1, wherein the second expandable member is configured to limit movement of the first expandable member within the body of the patient along a longitudinal axis of the first expandable member.

3. The apparatus of claim 1, wherein:
the portion of the bone is a first portion; and
the second expandable member is configured to engage a second portion of the bone such that a location of the first expandable member is maintained, the second portion of the bone being different from the first portion of the bone.

4. The apparatus of claim 1, wherein:
the bone is a vertebral body;
the portion of the bone is a cancellous portion of the vertebral body;
the first expandable member is configured to be disposed within the vertebral body when in its collapsed configuration; and
the first expandable member is configured to compress the cancellous portion of the vertebral body when moved from its collapsed configuration to its expanded configuration.

5. The apparatus of claim 1, wherein the first expandable member is constructed from a first material and the second expandable member is constructed from a second material different from the first material.

6. The apparatus of claim 1, wherein the first expandable member is disposed apart from the second expandable member such that the first expandable member does not contact the second expandable member when the first expandable member and the second expandable member are in their respective expanded configurations.

7. The apparatus of claim 1, wherein:
the first expandable member is coupled to a catheter in a first longitudinal position; and
the second expandable member is coupled to the catheter in a second longitudinal position different than the first longitudinal position.

8. The apparatus of claim 1, wherein:
the first expandable member is coupled to a catheter in a first circumferential position; and
the second expandable member is coupled to the catheter in a second circumferential position different than the first circumferential position.

9. The apparatus of claim 1, wherein the first expandable member is disposed within the second expandable member.

10. The apparatus of claim 1, wherein the first expandable member defines an interior region and the second expandable member defines an interior region, the interior region of the second expandable member being fluidically isolated from the interior region of the first expandable member.

11. An apparatus comprising:
a catheter;
a first expandable member coupled to the catheter, the first expandable member having a collapsed configuration and an expanded configuration, the first expandable member configured to displace a portion of a bone when moved from its collapsed configuration to its expanded configuration; and
a second expandable member coupled to the catheter, the second expandable member having a collapsed configuration and an expanded configuration, the second expandable member configured to limit movement of the catheter within a body of a patient when the second expandable member is in its expanded configuration,
the second expandable member configured to be expanded independently from the first expandable member.

12. The apparatus of claim 11, wherein a compliance the first expandable member is different from a compliance of the second expandable member.

13. The apparatus of claim 11, wherein:
the first expandable member is coupled to the catheter in a first longitudinal position; and
the second expandable member is coupled to the catheter in a second longitudinal position different than the first longitudinal position.

14. The apparatus of claim 11, wherein:
the first expandable member is coupled to the catheter in a first longitudinal position; and
the second expandable member is coupled to the catheter in a second longitudinal position; and
the catheter is configured to selectively adjust a distance between the first longitudinal position and the second longitudinal position.

15. The apparatus of claim 11, wherein:
the catheter includes a first shaft and a second shaft, the first shaft being movably disposable within the second shaft;
the first expandable member is coupled to the first shaft; and
the second expandable member is coupled to the second shaft.

16. An apparatus comprising:
a catheter defining an inflation lumen;
a first expandable member defining an interior region;
a second expandable member defining an interior region and having a collapsed configuration and an expanded configuration, the interior region of the second expandable member being fluidically isolated from the interior region of the first expandable member, the second expandable member configured to maintain a position of the first expandable member within a body of a patient when in its expanded configuration; and
a valve configured to selectively place the inflation lumen in fluid communication with the interior region of the first expandable member and the interior region of the second expandable member while maintaining fluid isolation between the interior region of the first expandable member and the interior region of the second expandable member.

17. The apparatus of claim 16, wherein the first expandable member has a collapsed configuration and an expanded configuration, the first expandable member being configured to displace a portion of a bone when moved from its collapsed configuration to its expanded configuration.

18. The apparatus of claim 16, wherein a compliance the first expandable member is different from a compliance of the second expandable member.

19. The apparatus of claim 16, wherein:
the first expandable member is coupled to the catheter in a first longitudinal position; and
the second expandable member is coupled to the catheter in a second longitudinal position different than the first longitudinal position.

20. The apparatus of claim 16, wherein:
the catheter includes a first shaft and a second shaft, the first shaft being movably disposable within the second shaft;
the first expandable member is coupled to the first shaft; and
the second expandable member is coupled to the second shaft.

21. The apparatus of claim 16, wherein:
the first expandable member is coupled to the catheter in a first longitudinal position; and
the second expandable member is coupled to the catheter in a second longitudinal position; and
the catheter is configured to selectively adjust a distance between the first longitudinal position and the second longitudinal position.

22. A method, comprising:
inserting into a body a catheter having a first expandable member and a second expandable member;
moving the second expandable member from a collapsed configuration to an expanded configuration such that the second expandable member limits movement of the first expandable member relative to the body; and
moving the first expandable member from a collapsed configuration to an expanded configuration such that the first expandable member displaces a portion of a tissue.

23. The method of claim 22, wherein the inserting includes inserting at least a portion of the catheter into a vertebral body.

24. The method of claim 22, further comprising defining percutaneously a passageway within a body of a patient before the inserting, the inserting including inserting the catheter via the passageway.

25. the method of claim 22, wherein the moving the second expandable member includes moving the second expandable member to the expanded configuration such that the second expandable member engages a structure within the body.

26. The method of claim 22, wherein the moving the second expandable member is done independently from the moving the first expandable member.

27. The method of claim 22, further comprising selectively adjusting a longitudinal distance between the first expandable member and the second expandable member.

28. The method of claim 22, wherein the tissue is any one of a bone tissue, an end plate of a vertebra, or a disc-related tissue.

29. A method, comprising:
inserting into a body a catheter having a first expandable member and a second expandable member;
moving the second expandable member from a collapsed configuration to an expanded configuration such that the second expandable member contacts a first portion of a bone structure; and
moving the first expandable member from a collapsed configuration to an expanded configuration such that the first expandable member displaces a second portion of the bone structure.

30. The method of claim 29, wherein the moving the second expandable member is done independently from the moving the first expandable member.

31. The method of claim 29, further comprising:
moving the first expandable member relative to the second expandable member along a longitudinal axis of the catheter, after the moving the second expandable member.

32. The method of claim 29, further comprising:
rotating the first expandable member relative to the second expandable member about a longitudinal axis of the catheter, after the moving the second expandable member.

33. The method of claim 29, wherein the collapsed configuration of the first expandable member is a first collapsed configuration and the expanded configuration of the first expandable member is a first expanded configuration, the method further comprising:
moving the first expandable member from the first expanded configuration to a second collapsed configuration after the moving the first expandable member from the first collapsed configuration;
moving the first expandable member relative to the second expandable member after the moving the first expandable member from the first expanded configuration; and
moving the first expandable member from the second collapsed configuration to a second expanded configuration such that the first expandable member displaces a third portion of the bone structure, the third portion being different than the second portion.

34. The method of claim 29, wherein the second portion of the bone structure is different than the first portion of the bone structure.

35. A kit, comprising:
a catheter having a first expandable member configured to displace a first portion of a bone structure when moved from a collapsed configuration to an expanded configuration and a second expandable member configured to engage a second portion of the bone structure when in an anchor configuration such that movement of the first expandable member within a body of a patient is limited; and
a trocar configured to define a passageway within the body through which the catheter can be inserted to access the bone structure.

36. The kit of claim 35, further comprising a cannula configured to be disposed within the passageway, the cannula configured to receive at least a portion of the catheter.
